(19) United States
(12) Patent Application Publication
(10) Pub. No.: US 2013/0197064 A1
(43) Pub. Date: Aug. 1, 2013

(54) METHOD AND COMPOSITION FOR ALVEOLAR EPITHELIAL CELL-SPECIFIC NUCLEIC ACID NUCLEAR IMPORT

(71) Applicant: David A. Dean, Rochester, NY (US)

(72) Inventor: David A. Dean, Rochester, NY (US)

(73) Assignee: UNIVERSITY OF ROCHESTER, Rochester, NY (US)

(21) Appl. No.: 13/634,422

(22) PCT Filed: Mar. 14, 2011
(86) PCT No.: PCT/US11/28368

§ 371 Date: Dec. 7, 2012

Related U.S. Application Data
(60) Provisional application No. 61/313,471, filed on Mar. 12, 2010.

(51) Int. Cl. C12N 15/113; C12N 15/85; A61K 48/00

(52) U.S. Cl. CPC C12N 15/113; A61K 48/00; C12N 15/85
USPC 514/44 R; 536/24.1; 435/320.1; 435/252.33; 435/325; 435/455; 604/22; 604/501

(57) ABSTRACT

One aspect of the invention relates to an isolated nuclear targeting molecule that includes a fragment of a mammalian glycoprotein 36 (gp36, also known as T1-α or podoplanin) gene expressed in type I alveolar epithelial cells. Plasmids containing the isolated nuclear targeting molecule which are useful for affording nuclear uptake of the plasmid DNA in type I alveolar epithelial cells but not type II alveolar epithelial cells, and compositions and host cells containing such plasmids are also disclosed. Use of the plasmids for targeting an exogenous DNA into nuclei of type I alveolar epithelial cells is described herein.

The present application is entitled to priority benefit of U.S. Provisional Patent Application Ser. No. 61/313,471, filed Mar. 12, 2010, which is hereby incorporated by reference in its entirety.

### FIELD OF THE INVENTION

The subject invention is directed to a molecule and method for importing DNA into the nuclei of a type I alveolar epithelial cells.

### BACKGROUND OF THE INVENTION

To date, only three approaches to deliver agents to specific cell and/or tissues types have been used to any extent: (1) application of the delivery agent to a desired cell type or tissue (e.g., injection of DNA into the anterior chamber of the eye for delivery to the cornea as opposed to other tissues); (2) use of cell surface receptor-ligand interactions (e.g., incorporation of folate into liposomes for delivery to folate receptor-expressing tumor cells); and (3) use of cell-specific promoters to drive transcription (e.g., use of the smooth muscle alpha actin promoter to limit transgene expression to smooth muscle cells). Each has advantages and disadvantages, but the reliance on just three methods is insufficient given the current needs to study mechanisms regulating development, specific cell heterogeneities and phenotypes, cell biology, and pathogenesis, as well as treat and image disease states without affecting surrounding cells and tissues.

Over the past several years a fourth, novel and robust method has been developed for cell-specific delivery of DNA. Using a variety of approaches, including transfection, microinjection, permeabilized cell systems, and animal models, it has been demonstrated that plasmid nuclear import in non-dividing cells is a sequence-specific process that requires the cytoplasmic binding of transcription factors to these DNA sequences (Dean, “Import of Plasmid DNA into the Nucleus is Sequence Specific,” Exp Cell Res 230(2):293-302 (1997); Dean et al., “Sequence Requirements for Plasmid Nuclear Import,” Exp Cell Res. 253(2):713-22 (1999); Wilson et al., “Nuclear Import of Plasmid DNA in Digitonin-permeabilized Cells Requires Both Cytoplasmic Factors and Specific DNA Sequences,” J Biol Chem 274(31):22025-32 (1999); Mesika et al., “A Regulated, NFκB-assisted Import of Plasmid DNA into Mammalian Cell Nuclei,” Mol Ther. 3(5 Pt 1):653-7 (2001); Goncalves et al., “An Optimized Extended DNA kappa B Site that Enhances Plasmid DNA Nuclear Import and Gene Expression,” J Gene Med 11:401-11 (2009)). Sequences have been identified that act in all cell types, due to the binding of ubiquitously expressed transcription factors, as well as sequences that direct DNA nuclear entry in specific cell types, due to binding of cell-specific transcription factors (Miller et al., “Tissue-specific and Transcription Factor-mediated Nuclear Entry of DNA,” Adv Drug Deliv Rev 61(7-8):603-13 (2009)). Transcription factors spend a large amount of their life in the cytoplasm, either after synthesis or due to regulation, and contain nuclear localization sequences (NLSs) or associate with other proteins that contain NLSs for their nuclear entry.

Under normal circumstances, a typical transcription factor would be transported into the nucleus, bind to its DNA target sequence present in various promoters, and activate or repress transcription. However, if a plasmid containing the transcription factor binding site is present in the cytoplasm, the transcription factor(s) in the cytoplasm can bind to this site before nuclear import, resulting in a plasmid coated with one or more NLSs. The NLS import machinery will then bind to the DNA-bound transcription factors and translocate the DNA-protein complex into the nucleus in the absence of cell division (Wilson et al., “Nuclear Import of Plasmid DNA in Digitonin-permeabilized Cells Requires Both Cytoplasmic Factors and Specific DNA Sequences,” J Biol Chem 274(31):22025-32 (1999); Miller et al., “Cell-specific Nuclear Import of Plasmid DNA in Smooth Muscle Requires Tissue-specific Transcription Factors and DNA Sequences,” Gene Ther. 15(15):1107-1115 (2008); Munkonge et al., “Identification and Functional Characterisation of Cytoplasmic Determinants of Plasmid DNA Nuclear Import,” J Biol Chem. 284:26978-26987 (2009)). It is important to note that only a small number of promoters or enhancers act as DNA nuclear targeting sequences, since although a number of transcription factors may bind to any promoter, many of the NLSs will reside at the DNA-binding surface or at protein-protein interfaces and will not be surface exposed and accessible to the NLS receptors. To date, cell-specific DNA targeting sequences that act in smooth muscle cells, osteoblasts, endothelial cells, and alveolar type 2 epithelial cells have been identified. These sequences support DNA nuclear import and subsequent gene expression from the plasmids only in their respective cells in vitro and in living animals.

No DNA targeting sequence has been identified for alveolar type 1 epithelial cells. Because alveolar type 1 epithelial cells make up 95% of the surface area of the lung, it would be extremely useful to identify a DNA targeting sequence that is specific for these cells.

The present invention is directed to overcoming the above-noted deficiency in the art.

### SUMMARY OF THE INVENTION

A first aspect of the invention relates to an isolated nuclear targeting molecule that includes a fragment of a mammalian glycoprotein 36 (gp36, also known as T1-α or podoplanin) gene expressed in type I alveolar epithelial cells.

Preferably, the fragment from the mammalian glycoprotein 36 (gp36) comprises the nucleotide sequence of SEQ ID NO: 1, SEQ ID NO: 2, or SEQ ID NO: 3, as well as fragments thereof which are effective for nuclear targeting thereof.

A second aspect of the invention relates to a plasmid for targeting an exogenous DNA molecule into nuclei of type I alveolar epithelial cells, the plasmid including a nuclear targeting molecule according to the first aspect of the invention, which affords nuclear uptake of the plasmid DNA in type I alveolar epithelial cells but not type II alveolar epithelial cells; and a restriction enzyme cleavage site that is suitable for insertion of an exogenous DNA to be targeted to the nuclei of type I alveolar epithelial cells.

According to one preferred embodiment of the invention, the plasmid includes an exogenous DNA molecule inserted into the restriction enzyme cleavage site, and a promoter region upstream (i.e., to the 5′ side) of the cleavage site or the exogenous DNA inserted therein.

A third aspect of the invention relates to an isolated host cell that includes a plasmid according to the second aspect of the invention.

A fourth aspect of the invention relates to a composition that includes a pharmaceutically acceptable carrier; and a plasmid according to the second aspect of the invention.

A fifth aspect of the invention relates to a method of targeting an exogenous DNA into nuclei of type I alveolar epithelial cells. This method includes: providing a plasmid according to the second aspect of the invention; and introducing the plasmid into the cytoplasm of type I alveolar epithelial cells, wherein the nuclear targeting molecule targets the exogenous DNA into the nuclei of the type I alveolar epithelial cells.

As demonstrated in the accompanying Examples, a new sequence is disclosed that promotes cell-specific nuclear import of plasmids in type I alveolar epithelial cells. Type I alveolar epithelial cells line 95% of the surface area of the lung and are responsible for most gas exchange between air and blood. They also play the major role in fluid homeostasis in the lung, maintaining appropriate lining fluid levels and removing pulmonary edema in disease states. Briefly, the Examples describe the screening of several promoters (DNA sequences containing binding sites for general and cell-specific transcription factors) that are expressed preferentially in type I alveolar epithelial cells for nuclear import activity. It is demonstrated that a 1352 nt sequence (SEQ ID NO: 1) containing the proximal promoter from the T1-α (gp36 or podoplanin) gene can cause plasmids to enter the nuclei of type I cells but not other cell types in the lung. Further, truncation studies demonstrate that fragments of SEQ ID NO: 1 will also afford nuclear import activity in type I alveolar epithelial cells. Based on these results, it is believed that corresponding results can be achieved using homologous promoter regions from other mammalian T1-α (gp36 or podoplanin) genes, such as from SEQ ID NO: 2 and SEQ ID NO: 3 and fragments thereof.

### BRIEF DESCRIPTION OF THE DRAWINGS

FIGS. 1A-B illustrate a model for cell-specific DNA nuclear import. Certain DNA nuclear Targeting Sequences have been shown to act in cell-restricted manners. In the case of certain “Cell type X” promoters, which act as Cell type X-specific DTSs, it is believed that the cell-specific transcription factors, TF-X1 and TF-X2, form complexes with the plasmid leading to an importin-recognizable complex that can be localized to the nucleus (FIG. 1A). By contrast, in all other cell types that do not express one or the other of these factors, an importin-binding complex is not formed leading to greatly reduced nuclear import (FIG. 1B). As demonstrated in the accompanying Examples, this model is valid in type 1 alveolar epithelial cells.

FIG. 2 is a fragment of the rat TP1α (gp36 or podopladin 1) gene that includes a portion of the promoter region (−1251 to −1) and exon 1 (+1 to +101).

FIG. 3 is a fragment of the human TP1α (gp36 or podopladin 1) gene that includes a portion of the promoter region (−1872 to −1) and exon 1 (+1 to +205).

FIG. 4 is a fragment of the rhesus macaque TP1α (gp36 or podopladin 1) gene that includes a portion of the promoter region and exon 1. The fragment was obtained from Genbank Accession AC191986 (from nt 171663 to 173500), which is hereby incorporated by reference in its entirety. The rhesus sequence shares ˜93% identity to nt 238 to 2077 of SEQ ID NO: 2.

FIGS. 5A-B illustrate two embodiments of “empty” plasmids of the invention. One empty plasmid of the invention (FIG. 5A) contains a restriction enzyme cleavage region and a DNA molecule that imparts nuclear uptake in only type 1 alveolar epithelial cells. Another empty plasmid of the invention (FIG. 5B) contains these same regions, as well as a promoter-effective DNA molecule located upstream of the restriction enzyme cleavage region.

FIG. 6 is a series of immunofluorescence images illustrating the characterization of type I-like AEC cells. Primary rat ATII cells were isolated and maintained on plastic for 7 days. Immunofluorescence was performed on Days 3, 5 and 7 for expression levels of ATI and ATII markers. The cell line R3/1 was also analyzed for expression of ATI and ATII markers.

FIG. 7 is a series of immunofluorescence images illustrating the nuclear import activity of plasmids containing the T1α promoter in R3/1 and primary day 5 type II cells. R3/1 (top) and day 5 primary rat type II (bottom) cells were cytoplasmically injected with plasmids containing the indicated promoters. 4.5 hours later, the location of the DNA was examined by fluorescence microscopy for Cy3-PNA (red).

FIG. 8 is a series of immunofluorescence images confirming that plasmids containing the T1α promoter show Type I cell-specific nuclear import activity. Cy3-labeled plasmids carrying the 1.3 kb T1α promoter, the AQP-5 promoter, the SV40 promoter, or no nuclear import sequence were microinjected into the cytoplasm of a type II cell line, primary rat type II cells, or bronchial smooth muscle cells. Four hours later, nuclear import was evaluated. The T1α promoter did not support nuclear import in any of these cell types.

FIG. 9 is a schematic illustrating putative transcription factor binding sites within SEQ ID NO: 1. The binding sites are designated symbolically. There are seven putative CEBPα-binding sites, two putative NF-κB binding sites, twelve putative Sp1 binding sites, a single putative AP-1 binding site, three putative GATA-1 binding sites, two putative CEBPβ binding sites, two putative NF1 binding sites, four putative AP-2 binding sites, one putative HNF3 binding site, one putative TTF-1 binding site, and one putative TGT3 binding site.

FIG. 10 illustrates the results of truncation studies to identify a minimal nuclear localization signal in the T1α promoter of SEQ ID NO: 1. Eight truncation constructs were prepared as shown to define the required regions of the nuclear localization sequence. These constructs were introduced into plasmids, and then microinjected into the cytoplasm of primary type I alveolar epithelial cells. Eight hours later, the subcellular localization of the plasmids was determined.

### DETAILED DESCRIPTION OF THE INVENTION

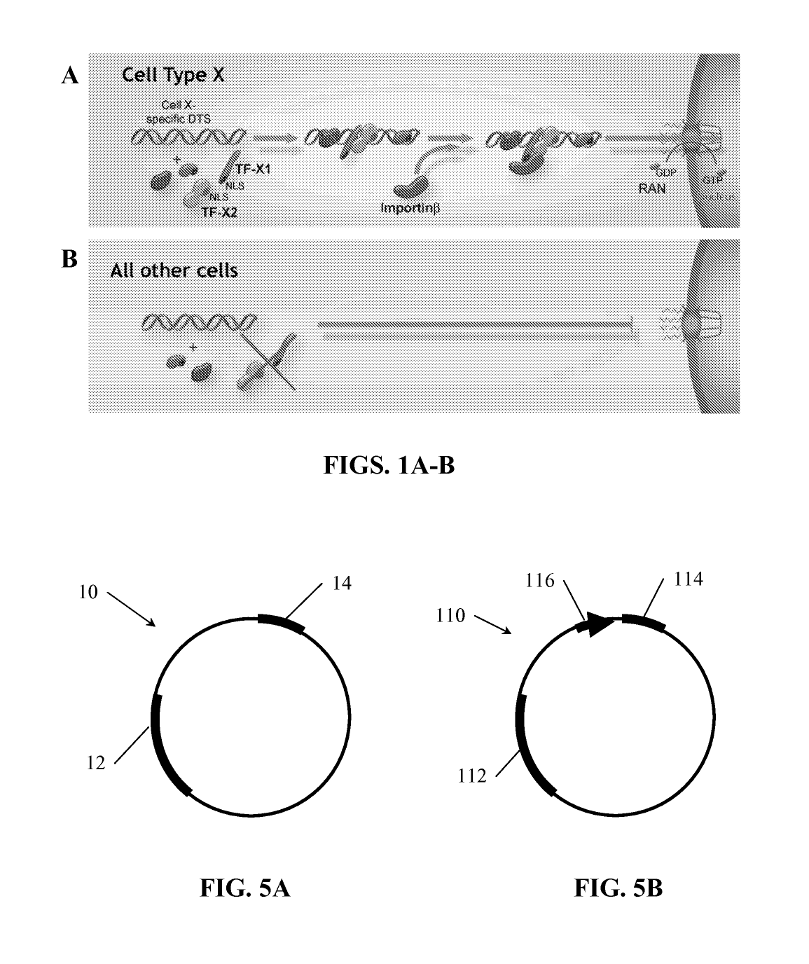

The invention provides a molecule and method for cell-specific nuclear import of DNA. This import is mediated by sequences containing binding sites for nuclear DNA binding proteins, such as eukaryotic transcription factors, DNA replication factors, and telomere and centromere binding proteins. Since nuclear DNA binding proteins bind to specific DNA sequences and contain or complex with nuclear localization signals (NLSs) for their nuclear import, it is believe that these proteins likely “coat” the DNA with NLSs, thereby allowing the DNA to utilize the NLS-mediated import machinery for nuclear entry (see FIG. 1A). By constructing a plasmid that contains the binding site for a nuclear DNA binding protein (for example, a transcription factor) that is expressed in a type I alveolar epithelial cell but not in any other cell type, the plasmid and its DNA payload is targeted to the nucleus only in the type I alveolar epithelial cell. In regard to the model in FIG. 1A, the cell-specific transcription factors of the type I alveolar epithelial cell binding sites are in the piece of DNA that is targeted to the nucleus.

According to the subject invention, a plasmid has been constructed for cell-specific import into the nuclei of type I alveolar epithelial cells using DNA sequence elements from a promoter of a mammalian gp36 (or T1α or podoplanin) gene, which is expressed only in type I alveolar epithelial cells. Nuclear import of DNA containing elements for this promoter occurs only in type I alveolar epithelial cells and no other cell types of the lung, including type II alveolar epithelial cells. Based on this finding, a new generation of DNA plasmid vectors that target to type I alveolar epithelial cells has been designed, and they can be used in any desired gene therapy directed to type I alveolar epithelial cells. The presence of these sequences causes the vector DNA to migrate to the nucleus of type I alveolar epithelial cells, and therefore is particularly useful for delivery to the lungs.

As used herein, “cell-specific” means that the nuclear targeting molecule targets DNA to the nuclei of only type I alveolar epithelial cells and not to the nuclei of other cell types.

As also used herein, “nuclear DNA binding proteins” refer to DNA binding proteins that reside in the nucleus. These nuclear, DNA binding proteins are characterized in that they bind to short DNA sequences with sequence specificity, and they are transported to the nucleus of a cell because they contain a nuclear localization signal (NLS) or because they complex with one or more other proteins that contain an NLS. These nuclear, DNA binding proteins have different functions in the regulation of DNA transcription and/or replication. Nuclear, DNA binding proteins include, for example, eukaryotic transcription factors, DNA replication factors, and telomere or centromere binding proteins. As also used herein, “transcription factors” refer to proteins that promote RNA polymerase recognition and/or initiation and/or activation and/or repression of promoters (DNA sequences). The binding of RNA polymerase to a promoter is necessary to initiate transcription, which is the process by which the information contained in the DNA is copied into a single-stranded RNA molecule by RNA polymerase. The genetic information present in an mRNA molecule is then translated into a protein.

Preferably, the nuclear DNA binding protein is a transcription factor. In one presently preferred embodiment, the specific cell type is a type I alveolar epithelial cell, and the binding site for a nuclear DNA binding protein is within the gp36 (or T1α or podoplanin) promoter and/or exon 1 region. An isolated gp36 nucleic acid molecule that excludes at least 90% of the coding region of gp36, more preferably at least 95% of the coding region is contemplated herein.

In certain embodiments, the cell-specific nuclear targeting molecule can have a nucleic acid sequence as shown in SEQ ID NO:1 or a cell-specific nuclear targeting portion thereof. Exemplary fragments include, without limitation, nt −1000 to +101 of SEQ ID NO: 1, nt −600 to +101 of SEQ ID NO: 1, and nt −200 to +101 of SEQ ID NO: 1

In certain other embodiments, the cell-specific nuclear targeting molecule can have a nucleic acid sequence as shown in SEQ ID NO: 2 or a cell-specific nuclear targeting portion thereof.

In further embodiments, the cell-specific nuclear targeting molecule can have a nucleic acid sequence as shown in SEQ ID NO: 3 or a cell-specific nuclear targeting portion thereof.

Corresponding DNA sequences from other mammalian gp36 can be isolated using standard BLAST searches of known mammalian genomes (e.g., rat, bull, horse, orangutan, rhesus monkey, canine), which are available on Genbank. For example, the rat and human gp36 sequences of SEQ ID NOS: 1 and 2 share ˜57% identity, within their region of overlap, as detected by Clustal alignment, and the human and rhesus gp36 sequences of SEQ ID NOS: 2 and 3 share ˜93% identity, within their region of overlap, as detected by Clustal alignment.

As used herein, a nucleic acid sequence which has a sequence as shown in a particular SEQ ID NO refers to a nucleotide sequence which has substantially the same nucleotide sequence, i.e., having at least 50% nucleotide identity, more preferably at least 70% identity, 80% identity, 90% identity, or 95% identity. Nucleotide additions, deletions, and/or substitutions which do not alter the functional characteristic of the molecule are encompassed by a nucleic acid sequence which is as shown in a particular SEQ ID NO, i.e., the resulting molecule is capable of cell-specific targeting of a DNA molecule to the nuclei of a type I alveolar epithelial cell. As will be readily understood by those skilled in the art, numerous nucleotides in a SEQ ID NO are likely to be filler or spacer nucleotides which are not critical to function. An A or G which is such a filler or spacer nucleotide could thus readily be interchanged with a C or T, for example, without affecting the function of the molecule. Such nucleotides could also readily be deleted. A particular SEQ ID NO, as exemplified herein, is the gp36 (or T1α or podoplanin) promoter (or a nuclear targeting portion thereof) which includes the binding site for a nuclear DNA binding protein. Additional nucleotides 5′ or 3′ to the SEQ ID NO in the gp36 promoter (or portion thereof) could be added to the SEQ ID NO without detracting from the molecule's cell-specific nuclear targeting function. Such additions, deletions, and substitutions could be made by methods known in the art, including site directed mutagenesis. The cell-specific nuclear targeting molecule as claimed herein to have a particular SEQ ID NO is intended to cover such variations which do not alter function.

The nuclear targeting molecule from the gp36 promoter is a DNA molecule, and can be isolated from cells or synthetically constructed based on the desired nucleotide sequence. As used herein, the term “isolated” when used in conjunction with the gp36 promoter refers to a nucleic acid sequence separated from the entire cell genome or from another vector which includes the desired portion of the cell genome or from the remainder of the gp36 gene.

The nuclear targeting molecule is most readily used by providing a plasmid (an extrachromosomal piece of DNA) for targeting a DNA molecule of interest into a nucleus of a specific cell type. As shown in FIG. 5A, the empty plasmid 10, in its most basic form, includes a nuclear targeting molecule 12 derived from gp36, which affords nuclear uptake of plasmid DNA in type I alveolar epithelial cells but not type II alveolar epithelial cells, and a restriction site region 14 (preferably containing two or more restriction sites) that allows for introduction of a transgene or other DNA molecule to be targeted to the nuclei of type I alveolar epithelial cells. The structural connection between the nuclear targeting molecule 12 and the restriction site region 14 is that the two are contained on the same plasmid 10. Since the transgene or DNA molecule to be targeted need not be under expressional control of the nuclear targeting molecule, the DNA molecule does not need to be “downstream” of the promoter region of the nuclear targeting molecule. As should be readily understood by those skilled in the art, “upstream” and “downstream” refer to location in the plasmid relative to the orientation of a gene (the DNA molecule to be targeted). For example, if a gene is presented in a 5′ to 3′ orientation, sequences to the 5′ region of the gene are “upstream” and sequences to the 3′ region of the gene are “downstream”. In the case of a circular DNA molecule, upstream and downstream are given meaning in relation to a given gene.

As shown in FIG. 5B, the empty plasmid 110, in its most basic form, includes a nuclear targeting molecule 112 derived from gp36, which affords nuclear uptake of plasmid DNA in type I alveolar epithelial cells but not type II alveolar epithelial cells, a restriction site region 114 (preferably containing two or more restriction sites), and a promoter-effective DNA molecule 116 capable of inducing expression of any downstream coding region (inserted into the restriction site region 114). The promoter-effective DNA molecule 116 should be proximate to the restriction site region 114 such that the promoter-effective DNA molecule 116 is capable of driving transcription of the coding region of any DNA molecule that is inserted into the restriction site region 114. The structural connection between (i) the nuclear targeting molecule 12 and (ii) both the promoter-effective DNA molecule 116 and the restriction site region 14 is that these regions are contained on the same plasmid 110.

The DNA molecule to be introduced into the plasmid (and targeted to the nucleus) generally encodes a protein or functional RNA molecule which would be desirable to express in the nucleus of the type I alveolar epithelial cell, and generally is exogenous DNA (i.e., such an encoded protein or enzyme is not being expressed in the specific cell type or is being expressed at very low levels). Many examples of DNA molecules for which it would be desirable to import the molecules into a specific cell type should be readily apparent to those skilled in the art. For example, many proposed gene therapy techniques would benefit from the ability to import a DNA molecule into the nucleus according to the subject invention. In recent years, numerous examples of DNA molecules which could be imported according to the subject invention have been published. The following are examples, for illustration only, of suitable DNA molecules:
(i) cystic fibrosis transmembrane conductance regulator in pulmonary epithelia may be useful in the treatment and/or prevention of cystic fibrosis lung disease (Wagner et al., “Toward Cystic Fibrosis Gene Therapy,” Annu. Rev. Med. 48:203-216 (1997), which is hereby incorporated by reference in its entirety);
(ii) CAT1 or ARG1 inhibitors, such as RNAi, under control of an SpB or CCIO promoter, for treatment of asthma, airway hyperresponsiveness, chronic airway remodeling, chronic destructive pulmonary disease, and arthritis (U.S. Patent Application Publ. No. 20090156538, which is hereby incorporated by reference in its entirety);
(iii) α1-anti-trypsin, sodium-potassium-adenosine triphophatase, CF transmembrane conductance regulator, interleukins, etc., under control of a ubiquitin C promoter for treatment of cystic fibrosis, asthma, emphysema, pulmonary odema, and lung cancer (U.S. Patent Application Publ. No. 20040047846, which is hereby incorporated by reference in its entirety);
(iv) cyclooxygenase (COX-1) gene to increase production of prostacyclin and PGE₂ by the lungs and inhibit endotoxin induced pulmonary hypertension and edema for treatment of acute lung injury (Brigham et al., “Gene Therapy for Acute Lung Injury,” Intensive Care Med. 26(13):S119-S123 (2000), which is hereby incorporated by reference in its entirety); and
(v) angiopoietin-1 for treatment of acute lung injury (McCarter et al., “Cell-based Angiopoietin-1 Gene Therapy for Acute Lung Injury,” Am. J. Resp. Crit. Care Med. 175:1014-1026 (2007), which is hereby incorporated by reference in its entirety).
   Each of the above references provides a separate example of the applicability of the subject invention to nuclear importation of many different DNA molecules, for many different reasons. Although some of the cited art teaches the use of lung-specific promoters, it should be appreciated by persons of skill in the art that lung-specific promoter are not required in the present invention, because nuclear uptake is limited to the target cells of interest, namely type I alveolar epithelial cells. However, the lung-specific promoters can be used instead of constitutive promoters operable in all cell types. As should be readily apparent from the above examples, many applications of the method of the subject invention are in the area of gene therapy, where a protein or RNA of interest can be imported into the nuclei of the desired specific cell type.

Examples of RNA to be expressed include, without limitation, an “antisense oligonucleotide” that could inhibit the translation or stability of a cellular mRNA (e.g., siRNA, shRNA, miRNA), or a stable RNA such as a tRNA, a rRNA, a UsnRNA (involved in mRNA splicing), or 7SL RNA which is part of the signal recognition particle (SRP) for protein translocation into the endoplasmic reticulum. Antisense RNAs are very popular for their potential to alter cellular mRNA levels for desired genes. Another example would be “ribozymes”, RNAs that repair mutant mRNAs.

The plasmids 10, 110 of the subject invention may contain other elements in addition to the nuclear targeting molecule and the DNA molecule to be targeted. For example, it may be desirable to include a bacterial origin of replication (such as on C for replication in Escherichia coli, or the origin of replication of Bacillis subtilis for replication therein, or the origin of replication of Pseudomonas aeruginosa for replication therein, etc.) so that the plasmid can be maintained and replicated in a bacterial host. Such an embodiment of the plasmid of the subject invention could also include a selection marker for selecting bacterial colonies which contain the subject plasmid. Such selection or biological markers are well known in the art. In bacteria, these are commonly used drug-resistance genes. Drug or antibiotic resistance is used to select bacteria that have taken up cloned DNA from the much larger population of bacteria that have not.

A selection marker can also be included in the plasmid to identify mammalian cells which have taken up the plasmid DNA. In the early mammalian gene transfer experiments involving viral genes, the transfer of exogenous DNA into cells was detected because the DNA had a biological activity; it led to production of infectious virus or produced stable changes in the growth properties of the transfected cells. The herpes simplex virus thymidine kinase (HSV tk) gene can be used as a selectable genetic marker in mammalian cells in much the same way that drug-resistance genes work in bacteria, to allow rare transfected cells to grow up out of a much larger population that did not take up any DNA. The cells are transferred to selective growth medium, which permits growth only of cells that took up a functional tk gene (and the transferred DNA of interest). Various dominant selectable markers are now known in the art, including:
(i) aminoglycoside phosphotransferase (APH), using the drug G418 for selection which inhibits protein synthesis; the APH inactivates G418;
(ii) dihydrofo late reductase (DHFR):Mtx-resistant variant, using the drug methotrexate (Mtx) for selection which inhibits DHFR; the variant DHFR is resistant to Mtx;
(iii) hygromycin-B-phosphotransferase (HPH), using the drug hygromycin-B which inhibits protein synthesis; the HPH inactivates hygromycin B;
(iv) thymidine kinase (TK), using the drug aminopterin which inhibits de novo purine and thymidylate synthesis; the TK synthesizes thymidylate;
(v) xanthine-guanine phosphoribosyltransferase (XGPRT), using the drug mycophenolic acid which inhibits de novo GMP synthesis; XGPRT synthesizes GMP from xanthine;
(vi) adenosine deaminase (ADA), using the drug 9-β-D-xylofuranosyl adenine (Xyl-A) which damages DNA; the ADA inactivates Xyl-A; and
(vii) multidrug resistance (MDR), which is also known as the P-glycoprotein (Licht et al., “P-Glycoprotein-mediated Multidrug Resistance in Normal and Neoplastic Hematopoietic Cells,” Ann. Hematol. 69:159-171 (1994), which is hereby incorporated by reference in its entirety).

Gene amplification can also be used to obtain very high levels of expression of transfected genes. When cell cultures are treated with Mtx, an inhibitor of a critical metabolic enzyme, DHFR, most cells die, but eventually some Mtx-resistant cells grow up. A gene to be expressed in cells is cotransfected with a cloned dhfr gene, and the transfected cells are subjected to selection with a low concentration of Mtx. Resistant cells that have taken up the dhfr gene (and, in most cases, the co-transfected gene) multiply. Increasing the concentration of Mtx in the growth medium in small steps generates populations of cells that have progressively amplified the dhfr gene, together with linked DNA. Although this process takes several months, the resulting cell cultures capable of growing in the highest Mtx concentrations will have stably amplified the DNA encompassing the dhfr gene a hundredfold or more, leading to significant elevation of the expression of the co-transfected gene.

As noted above with respect to FIG. 5B, it may also be desirable to include, as an element of the plasmid according to the subject invention, a molecule encoding a promoter to control expression of the DNA molecule to be targeted. Such a promoter sequence would need to be positioned upstream from the DNA molecule to effectively control expression of the DNA molecule. RNA polymerase normally binds to the promoter and initiates transcription of a gene (the DNA molecule) or a group of linked genes and regulatory elements (operon). Promoters vary in their strength, i.e., ability to promote transcription. For the purpose of expressing the target DNA molecule, it is desirable to use strong promoters in order to obtain a high level of transcription and, hence, expression of the gene. The promoter could also be a lung-specific promoter, which only turns on in the lung tissue, or a developmentally regulated promoter which only turns on at a certain time in the development of a cell or tissue. Suitable promoters for expression of genes in animal cells include, for example, the beta-actin promoter, cytomegalovirus (CMV) promoter, Adenovirus major late promoter, Thymidylate kinase (TK) promoter, and the Rous Sarcoma Virus (RSV) LTR-promoter. It should be apparent that the additional promoter (to control expression of the target DNA molecule) should not be a ubiquitous promoter that includes non-cell specific binding sites for nuclear DNA binding proteins, as such a promoter may promote plasmid uptake in cells other than type I alveolar epithelial cells.

As indicated, the method of the subject invention involves the use of a plasmid vector. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These same techniques can be used to prepare the plasmids of the present invention, and then utilize the plasmids by introducing a target DNA molecule to be expressed in type I alveolar epithelial cells. Once the recombinant plasmids are produced, they can be introduced by means of transformation and replicated in prokaryotic and eukaryotic cells. The DNA sequences are cloned into the plasmid vector using standard cloning procedures known in the art, as described by Sambrook et al., MOLECULAR CLONING, Cold Spring Harbor Laboratory (1989), which is hereby incorporated by reference in its entirety.

Bacterial host cell strains and expression vectors can be chosen which inhibit the action of the promoter unless specifically induced. In certain operons the addition of specific inducers is necessary for efficient transcription of the inserted DNA; for example, the lac operon is induced by the addition of lactose or IPTG (isopropylthio-beta-D-galactoside). A variety of other operons, such as trp, pro, etc., are under different controls. The trp operon is induced when tryptophan is absent in the growth media; and the P_{L} promoter of lambda can be induced by an increase in temperature in host cells containing a temperature sensitive lambda repressor, e.g., c1857. In this way, greater than 95% of the promoter-directed transcription may be inhibited in uninduced cells. Thus, expression of the DNA molecule of the invention can be controlled.

When cloning in a eukaryotic host cell, enhancer sequences (e.g., the enhancer from the CMV immediate early promoter or the retroviral long terminal repeats of LTRs, etc.) may be inserted to increase transcriptional efficiency. Enhancer sequences are a set of eukaryotic DNA elements that appear to increase transcriptional efficiency in a manner relatively independent of their position and orientation with respect to a nearby gene. Unlike the classic promoter elements (e.g., the polymerase binding site and the Goldberg-Hogness “TATA” box) which must be located immediately 5′ to the gene, enhancer sequences have the remarkable ability to function upstream from, within, or downstream from eukaryotic genes. Therefore, the position of the enhancer sequence with respect to the inserted gene is less critical.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (SD) sequence about 7-9 bases 5′ to the initiation codon (ATG) to provide a ribosomal binding site. Thus, any SD-ATG combination that can be utilized by host cell ribosomes can be employed. Such combinations include but are not limited to the SD-ATG combination from the CRO gene or the N gene of coliphage lambda, or from the E. coli tryptophan E, D, C, B or A genes. Additionally, any SD-ATG combination produced by recombinant DNA or other techniques involving incorporation of synthetic nucleotides can be used.

In accordance with the subject invention, the DNA of the plasmid as described herein is targeted into the nuclei of the type I alveolar epithelial cell, where the DNA molecule to be targeted is expressed. Since the nuclear-localized plasmid DNA will eventually be degraded, it may be desirable for long term expression of the DNA molecule in the nuclei of the specific cell type to integrate the plasmid DNA into the genome of the specific cell type. In such an embodiment, the plasmid of the subject invention further includes a molecule to direct integration of the DNA molecule into the genome of the specific cell type. Such integration sequences are known in the art, and include, for example, the inverted terminal repeats of adeno-associated virus (ITRs), retroviral long terminal repeats (LTRs), and other viral sequences shown to cause incorporation or integration of the viral genome into the specific cell type genome.

As should be readily apparent, various additional elements can be included in the plasmid of the subject invention depending upon the desired goal. For ease in constructing various embodiments of the plasmid, the basic empty plasmid can contain a number of unique restriction enzyme sites for insertion of the various molecules or elements. As used herein, a “unique” restriction enzyme site refers to the presence of only one cleavage site for a particular restriction endonuclease within the plasmid DNA. That particular restriction endonuclease (or restriction enzyme) will, therefore, only cleave the DNA of the plasmid at that one location or “unique” site. These unique restriction sites can be provided in the plasmid of the subject invention by including a polylinker as an element of the plasmid. As used herein, a “polylinker” refers to a sequence which contains many restriction enzyme recognition sequences that are present only once in the vector or plasmid, i.e., unique restriction sites. The plasmid of the subject invention may also contain restriction sites that occur twice in close proximity (i.e., the flanking sites of the polylinker) and these could also be used to clone in sequence between the sites.

Having constructed the plasmid according to the subject invention, a host cell comprising the plasmid is also provided by the subject invention. As indicated above, for maintenance and propagation of the plasmid, a bacterial host cell (such as Escherichia coli) may be used. Bacterial host cells for maintenance and propagation offer the advantages of being easy to work with and capable of rapid reproduction and therefore propagation of the plasmid.

In use, however, the DNA molecule to be targeted to the nucleus of a type I alveolar epithelial cell is most likely to express a product useful in mammals. For example, and referring to the many possible uses of the subject invention discussed above, the host cell may be a type I alveolar epithelial cell. However, a host cell can also be a type of cell which is to reproduce the plasmid, such as a eukaryotic host.

A viral vector may provide the means for introducing the plasmid into the host cell. For example, the plasmid may be introduced into an adenovirus, retrovirus, adeno-associated virus, vaccinia virus, papovavirus, or herpes simplex virus vector and these viral vectors can then infect a mammalian cell in order to get the plasmid DNA into the cytoplasm and/or nucleus of the mammalian cell. Other mammalian viruses could similarly be used. Alternatively, the “naked” plasmid can be used in a suitable composition.

The nuclear targeting molecule of the subject invention also offers the advantage of being able to target a DNA molecule to the nucleus of a non-dividing type I alveolar epithelial cells. Non-dividing cells include two classes of cells: those that are not dividing (quiescent) and those that cannot divide (i.e., many terminally differentiated cell types). When cells leave mitosis and are finished dividing, they enter the G1 phase of the cell cycle and then come to a halt at G0 (G zero). At this point they are “growth-arrested”; protein synthesis is decreased as is transcription. Upon stimulation, most cells will exit G0 and continue on with the cell cycle, leading to division. However, many cells will remain in this G0 state for a long time. The period of quiescence for each type of cell is different, but if it is greater than a week, the method of the subject invention is especially applicable. For a general discussion of non-dividing cells, including quiescent and terminally differentiated cells, see Porth, PATHOPHYSIOLOGY: CONCEPTS OF ALTERED HEALTH STATES, 4th ed., JB Lippincott Co., Philadelphia, Pa., pp 72-74 (1994), which is hereby incorporated by reference in its entirety.

Having thus described the nuclear targeting molecule and plasmid according to the subject invention, as well as suitable host cells into which the plasmid can be introduced, the invention further provides a method of targeting a DNA molecule into the nuclei of a specific cell type. The method comprises first providing a plasmid according to the subject invention (which contains a DNA molecule to be expressed), and then introducing the plasmid into the cytoplasm of the type I alveolar epithelial cell. The nuclear targeting molecule, which is an element of the plasmid, targets the DNA molecule, which is another element of the plasmid, to the nuclei of the type I alveolar epithelial cell.

Various methods are known in the art for introducing nucleic acid molecules into host cells (including the specific cell type). One method is microinjection, in which DNA is injected directly into the cytoplasm of cells through fine glass needles. Alternatively, DNA can be incubated with an inert carbohydrate polymer (dextran) to which a positively charged chemical group (DEAE, for diethylaminoethyl) has been coupled. The DNA sticks to the DEAE-dextran via its negatively charged phosphate groups. Other polymer-based delivery vehicles can be used, including poly(ester amine) (Arote et al., “Biodegradable poly(ester amine)s for Gene Delivery Application,” Biomed. Mater. 4:044102(DOI) (2009); Arote et al., “A Biodegradable Poly(ester amine) Based on Polycaprolactone and Polyethyleneimine as a Gene Carrier,” Biomaterials 28(4):735-744 (2007); Guo et al., “Synthesis of Novel Biodegradable Poly(ester amine) (PEAs) Copolymer Based on Low-Molecular Weight Polyethyleneimine for Gene Delivery,” Int. J. Pharmaceutics 379(1):82-89 (2009), each of which is hereby incorporated by reference in its entirety) and poly(amido amine) (Piest et al., “Novel Poly(amido amine)s with Bioreducible Disulfide Linkages in Their Diamine Units: Structure Effects and in vitro Gene Transfer Properties,” J. Controlled Release 132(3):e12-13 (2008); Lin et al., “Novel Bioreducible Poly(amido amine)s for Highly Efficient Gene Delivery,” Bioconjugate Chem. 18(1):138-145 (2007), each of which is hereby incorporated by reference in its entirety). These large DNA-containing particles stick to the surfaces of cells, which are thought to take them in by a process known as endocytosis. In another method, cells efficiently take in DNA in the form of a precipitate with calcium phosphate. In electroporation, cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures (passage through these vesicles may sometimes destroy or damage DNA). Alternatively, naked plasmid can be introduced in a suitable saline solution, or nanoparticle-based systems. Enhancement of delivery to the type I alveolar epithelial cells can be accomplished using ultrasound contrasting agents with ultrasound (Unger et al., “Gene Delivery Using Ultrasound Contrasting Agents,” Echocardiography 18(4):355-361 (2003); Aneja et al., “Targeted Gene Delivery to the Lung,” Exp. Opin. Drug Delivery 6(6):567-583 (2009), each of which is hereby incorporated by reference in its entirety).

Further methods for introducing nucleic acid molecules into cells involve the use of viral vectors. Any suitable viral or infective transformation vector can be used. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, each of which is hereby incorporated by reference in its entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), each of which is hereby incorporated by reference in its entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, each of which is hereby incorporated by reference in its entirety.

To summarize, it has been shown that plasmid DNA containing a nuclear localization sequence from gp36 selectively transported into the nuclei of differentiated type I alveolar epithelial cell; and transport does not occur in any other cell type, particularly type II alveolar epithelial cells. Import occurs through the nuclear pore complex in the absence of mitosis, and is sequence-specific. A model has been developed in which import is mediated by sequences containing binding sites for eukaryotic transcription factors. Since transcription and replication factors bind to specific DNA sequences and contain nuclear localization signals (NLSs) for their nuclear import, these proteins “coat” the DNA with NLSs, thereby allowing the DNA to utilize the NLS-mediated import machinery for nuclear entry. Moreover, the cell-selective nuclear import of the gp36 sequence is mediated by transcription factors that are expressed exclusively in type I alveolar epithelial cell. These results are the first demonstration of cell-specific nuclear import of plasmid DNA into type I alveolar epithelial cells and allow the creation of new lung-specific gene therapy vectors that target the type I alveolar epithelial cells.

Based on the DNA sequences that are utilized for the nuclear import of plasmid DNA, a model for the import reaction (FIGS. 1A-B) is proposed. The DNA fragment of gp36 contains a region rich in consensus binding sites for numerous transcription factors. These may include one or more of AP1, AP2, NF-κB, SP1, GATA-1, DEBPα, CEBPβ, NF-1, HNF3, TTF-1, and TGT3. Since transcription factors, like all proteins, are translated in the cytoplasm, they must target to the nucleus either after synthesis or upon proper stimulation. To enter the nucleus they must either contain nuclear localization signals (NLSs) or form oligomers with other proteins that contain an NLS. Since transcription factors bind to specific DNA sequences, if DNA containing the appropriate sequences is present in the cytoplasm, it can be complexed by these proteins, thus coating the DNA with protein NLSs. The NLSs present in this nucleoprotein complex can then interact with the normal importin/karyopherin NLS receptor and enter the nucleus by the normal nuclear protein import machinery. The plasmid DNA is therefore imported into the nucleus where the target DNA molecule can be expressed.

Based on these findings, a new class of gene therapy vectors that are specific for delivery to the lungs is described herein, by virtue of the specificity for type I alveolar epithelial cells. Thus, any of a number of combinations can be made which lead to greatly improved gene therapy vectors that can (1) target to the nuclei of cells in the absence of cell division, and (2) do so in a cell-specific manner. An added advantage of this approach is that the use of vectors containing these types of cell-specific DNA targeting sequences will ensure safety since nuclear import and resulting gene expression will occur only in type I alveolar epithelial cells.

### EXAMPLES

The following examples are intended to illustrate practice of the invention, and are not intended to limit the scope of the claimed invention.

### Example 1

### Identification of Useful Cell Model

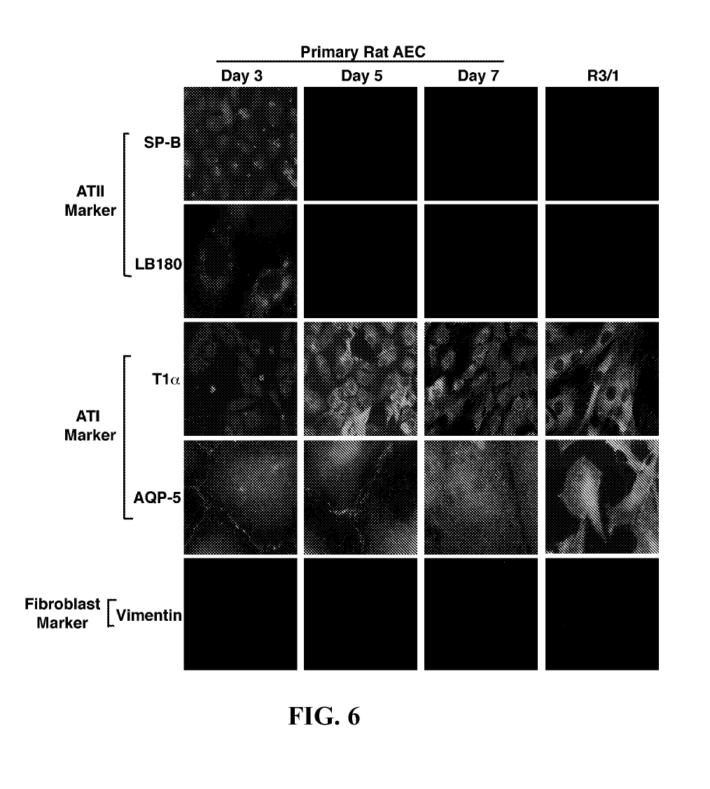

The study of ATI cells has been challenging due to the difficulty involved in isolating primary type I cells from animals as well as a limited selection of cell lines displaying type I characteristics. Consequently, many studies of ATI-specific promoters have been performed in cell lines representative of ATII cells, not ATI. While these results have proven informative, it is difficult to interpret their meaning in the context of an actual ATI cell. Thus, performance of these studies in cell types more representative of a type I cell rather than a type II cell was desired. It has previously been demonstrated that primary ATII cells cultured on plastic begin to take on a type I cell phenotype after several days in culture (Borok et al., “Modulation of T1alpha Expression with Alveolar Epithelial Cell Phenotype in vitro,” Am J Physiol 275(1 Pt 1):L155-64 (1998); Borok et al., “Keratinocyte Growth Factor Modulates Alveolar Epithelial Cell Phenotype in vitro: Expression of Aquaporin 5,” Am J Respir Cell Mol Biol 18(4):554-61 (1998); Campbell et al., “Caveolin-1 Expression and Caveolae Biogenesis During Cell Transdifferentiation in Lung Alveolar Epithelial Primary Cultures,” Biochem Biophys Res Commun. 262(3):744-51 (1999); Dobbs et al., “Changes in Biochemical Characteristics and Pattern of Lectin Binding of Alveolar Type II Cells with Time in Culture,” Biochim Biophys Acta 846(1):155-66 (1985); Dobbs et al., “Monoclonal Antibodies Specific to Apical Surfaces of Rat Alveolar Type I Cells Bind to Surfaces of Cultured, but not Freshly Isolated, Type II Cells,” Biochim Biophys Acta, 970(2):146-5 (1988), each of which is hereby incorporated by reference in its entirety). This process was characterized in the utilized system by demonstrating that primary rat ATII cells exhibit a type I expression pattern by day 5 (D5) in culture, as evidenced by the increase in expression of T1α and Aquaporin-5 as well as the loss of Surfactant Protein B (SP-B) and lamellar body 180 (LB180) expression (FIG. 6). Also, a rat cell line, R3/1, which has been previously reported to resemble ATI cells, was also utilized (Koslowski et al., “A New Rat Type I-like Alveolar Epithelial Cell Line R3/1: Bleomycin Effects on Caveolin Expression,” Histochem Cell Biol 121(6):509-19 (2004), which is hereby incorporated by reference in its entirety). In use, this cell demonstrated a highly type I cell phenotype (FIG. 6). Reverse transcriptase PCR for transcripts of all of these genes confirmed the immunofluorescence data. These data demonstrate that the R3/1 cell line and day 5 primary rat type II cells can be used as representative type I cells in further experiments.

### Example 2

### Demonstration That T1α Promoter Mediates Plasmid DNA Nuclear Import in Type I-Like Alveolar Epithelial Cells

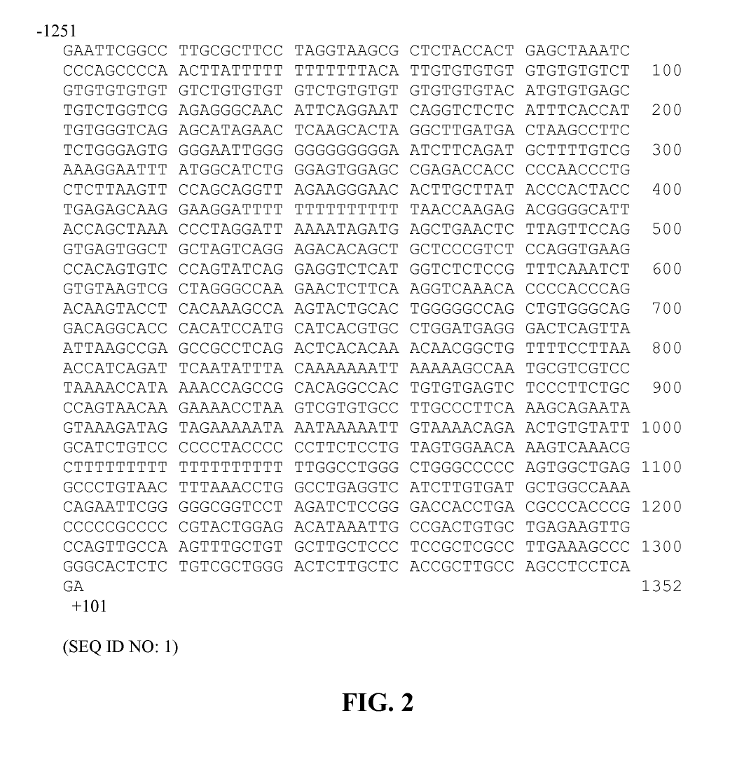

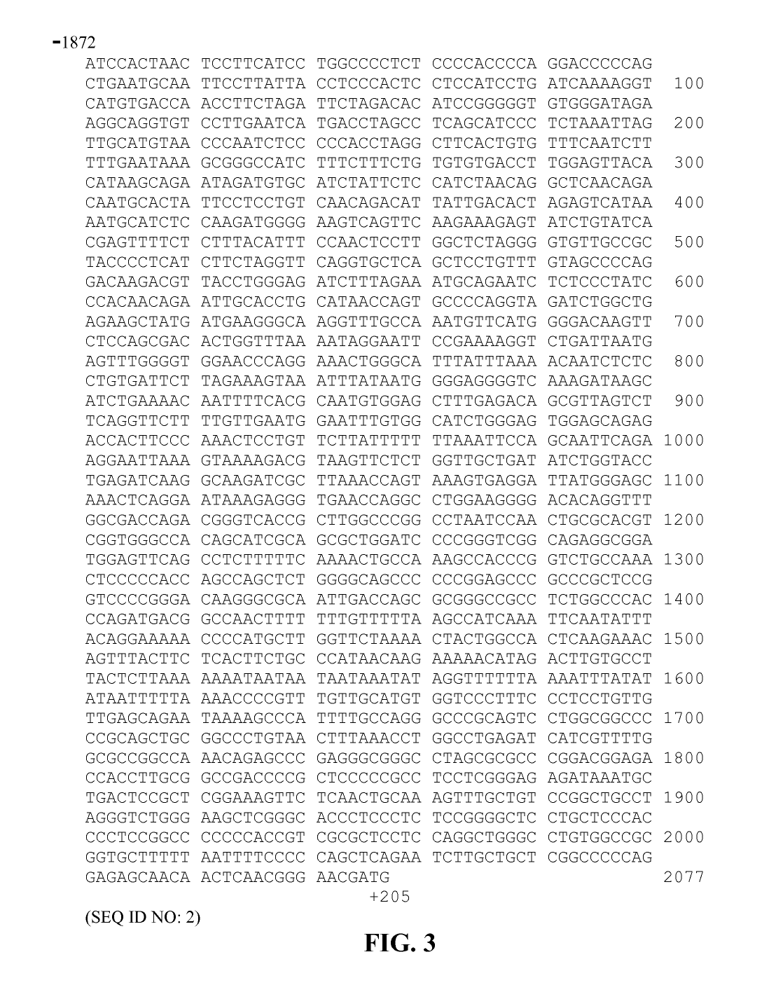

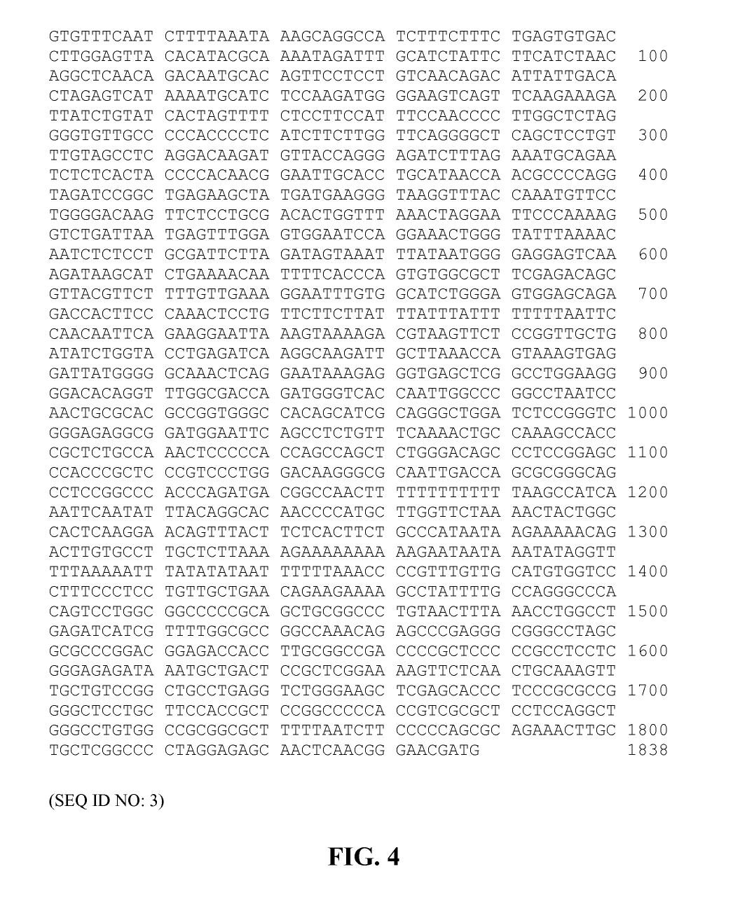

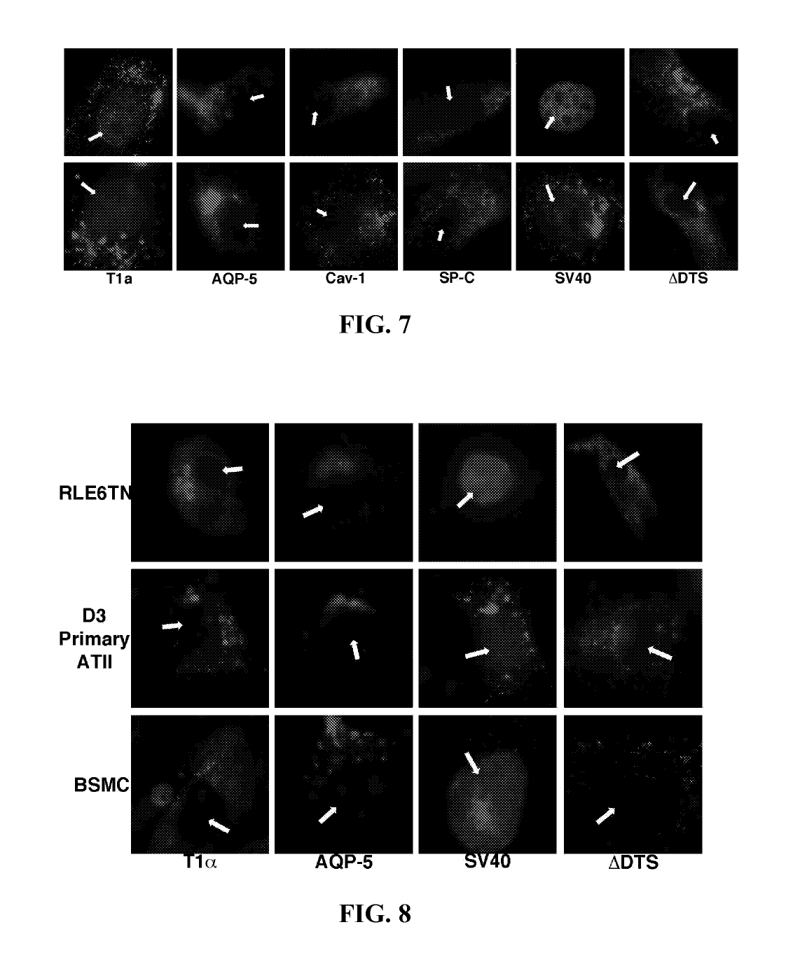

The T1α (−1251 to +101) (see FIG. 2), Aquaporin-5 (−1201 to +111) and Caveolin-1 (865 to +62) promoters were selected to study the potential presence of an ATI-specific DTS. The promoters were cloned into a GFP expression plasmid that contains no DNA nuclear import sequence, and nuclear import was assessed by cytoplasmically injecting these plasmids into either R3/1 cells, a rat cell line closely resembling type I cells, or primary rat type II cells which were cultured on plastic for 5 days (to give a type I phenotype). Cells were fixed at 4.5 hours post-injection, and the DNA was visualized via fluorescence microscopy for the Cy3-PNA labeled plasmids. As expected, when plasmid containing the SV40 DTS was injected into the cytoplasm of both cell types, greater than 90% of the DNA was present in the nucleus within 4.5 hours (FIG. 7). In contrast, the plasmid backbone of these plasmids, which does not contain a DTS, exhibited only cytoplasmic Cy3 signal after the incubation period. Interestingly, the plasmids containing the T1α promoter demonstrated nuclear import in both R3/1 and D5 primary type II cells (FIG. 7). While the effect was not as dramatic as the nuclear import seen with the SV40 DTS, a significant amount of import was observed in both cell types. The Aquaporin-5 promoter, however, produced variable results. In R3/1 cells, nuclear import of plasmids was observed within 4.5 hours. This event was not observed in D5 primary type II cells though. Plasmids carrying the Caveolin-1 promoter did not display any nuclear import in either cell types tested. These results demonstrate that the T1α promoter contains a DNA nuclear targeting sequence, which is able to mediate nuclear import in alveolar epithelial type I-like cells.

### Example 3

### Demonstration That T1α Promoter Does Not Mediate Plasmid Nuclear Import in Non-Type I Cells

To determine if the nuclear import activity of the T1α promoter was restricted to type I cells, plasmid containing this promoter, as well as plasmids containing the Aquaporin-5 and Caveolin-1 promoters, were microinjected into the cytoplasm of RLE6TN cells, a type II cell line, D3 primary rat type II cells, and Bronchial Smooth Muscle cells (BSMC). 4.5 hours post-injection, the cells were fixed and plasmid location was analyzed by visualization of the Cy3-PNA signal. All of these cells were also injected with plasmid containing the SV40 DTS to demonstrate that these cell types are able to import DNA in a sequence specific manner. As shown in FIG. 8, plasmids carrying the T1α promoter failed to be imported in any of the type II cells or the BSMC. This was also true for plasmids containing the Aquaporin-5 and Caveolin-1 promoters, as well as the promoterless plasmid (DDTS). These data demonstrate that the T1α promoter nuclear import activity is specific for type I cells.

### Example 4

### Truncation Analysis of SEQ ID NO: 1

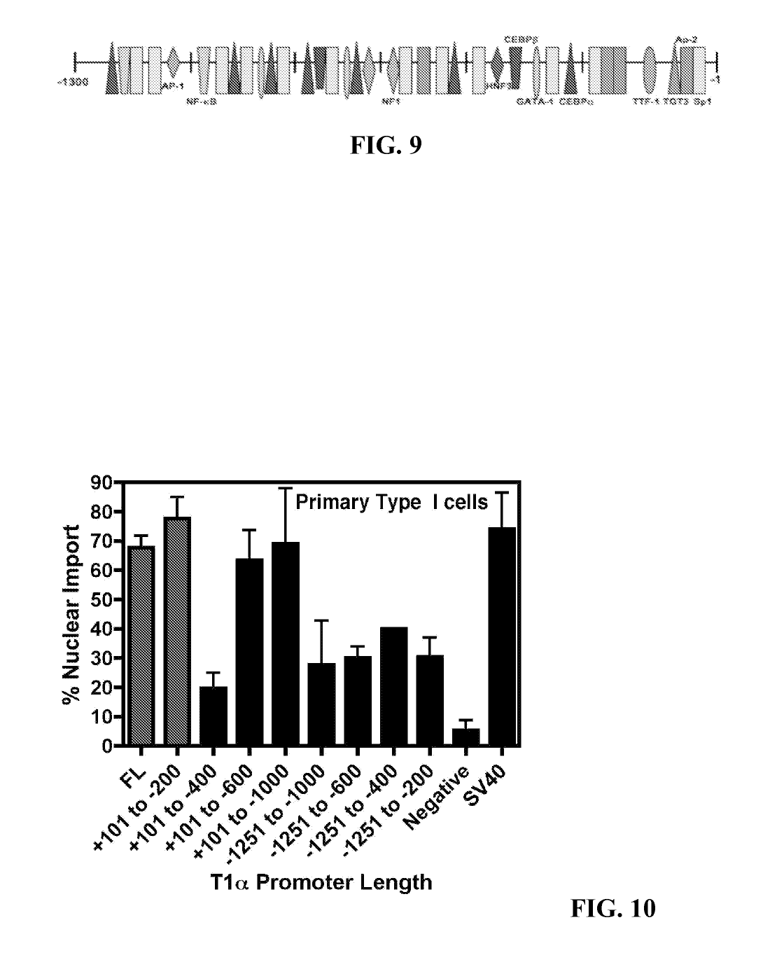

SEQ ID NO: 1 is represented diagrammatically in FIG. 9, with putative transcription factor binding sites designated symbolically. For example, there are seven putative CEBPα-binding sites, two putative NF-κB binding sites, twelve putative Sp1 binding sites, a single putative AP-1 binding site, three putative GATA-1 binding sites, two putative CEBPβ binding sites, two putative NF1 binding sites, four putative AP-2 binding sites, one putative HNF3 binding site, one putative TTF-1 binding site, and one putative TGT3 binding site.

Eight truncation constructs were prepared as shown in FIG. 10 to define the required regions of the nuclear localization sequence. These constructs were introduced into plasmids, and then microinjected into the cytoplasm of primary type I alveolar epithelial cells as described in Example 2 above. Eight hours later, the subcellular localization of the plasmids was determined via fluorescence microscopy for the Cy3-PNA labeled plasmids. The degree of fluorescence confirmed the percentage of nuclear import. The degree of fluorescence was measured by the total number of cells (out of 100) displaying nuclear fluorescence. These data demonstrate that the fragments of SEQ ID NO: 1 containing nucleotide sequences +101 to −200, +101 to −600, and +101 to −1000 have activity for nuclear import in type I cells as well as the full length +101 to −1251. Any of these truncations can be used to support DNA nuclear import in type I cells specifically, and it is believed that homologous fragments of SEQ ID NOS: 2 and 3 will also support nuclear import.

These fragments can be further defined by mapping the appropriate 200 bp or 400 bp region(s) into separate 25 bp or 50 bp regions (e.g., +101 to −100, +101 to −125, +101 to −150, +101 to −175, +101 to −225, +101 to −250, +101 to −275, +101 to −300 +101 to −425, +101 to −450, +101 to −475, +101 to −500, +101 to −525, +101 to −550, +101 to −570). Based on these truncation studies, it will be possible to identify which of the above-identified transcription factors mediate nuclear transport. Similar studies can be carried out using SEQ ID NO: 2 and SEQ ID NO: 3.

Although the invention has been described in detail for the purpose of illustration, it is understood that such detail is solely for that purpose, and variations can be made therein by those skilled in the art without departing from the spirit and scope of the invention which is defined by the following claims.

1. An isolated nuclear targeting molecule comprising a fragment of a mammalian glycoprotein 36 (gp36) gene expressed in type I alveolar epithelial cells.

2. The isolated nuclear targeting molecule according to claim 1, wherein the fragment of the mammalian glycoprotein 36 gene has the nucleotide sequence of SEQ ID NO: 1, SEQ ID NO: 2, or SEQ ID NO: 3.

3. (canceled)

4. The isolated nuclear targeting molecule according to claim 1, wherein the fragment of the mammalian glycoprotein 36 gene is a fragment of SEQ ID NO: 1, a fragment of SEQ ID NO: 2, or a fragment of SEQ ID NO: 3.

5. The isolated nuclear targeting molecule according to claim 4, wherein the fragment of SEQ ID NO: 1 comprises nt +101 to −200, +101 to −600, or +101 to −1000.

6-7. (canceled)

8. A plasmid for targeting an exogenous DNA molecule into nuclei of type I alveolar epithelial cells, the plasmid comprising:
a nuclear targeting molecule according to claim 1 that affords nuclear uptake of the plasmid DNA in type I alveolar epithelial cells but not type II alveolar epithelial cells; and
a restriction enzyme cleavage site that is suitable for insertion of an exogenous DNA to be targeted to the nuclei of type I alveolar epithelial cells.

9. The plasmid according to claim 8, further comprising an exogenous DNA to be targeted to the nuclei of type I alveolar epithelial cells, which is inserted into the plasmid at the restriction enzyme site.

10. The plasmid according to claim 9, further comprising a promoter operably coupled 5′ to the restriction enzyme cleavage site or the exogenous DNA.

11. The plasmid according to claim 8, wherein the fragment of the mammalian glycoprotein 36 gene has the nucleotide sequence of SEQ ID NO:1, SEQ ID NO: 2, or SEQ ID NO: 3.

12. (canceled)

13. The plasmid according to claim 8, wherein the fragment of the mammalian glycoprotein 36 gene is a fragment of SEQ ID NO: 1, a fragment of SEQ ID NO: 2, or a fragment of SEQ ID NO: 3.

14. The plasmid according to claim 13, wherein the fragment of SEQ ID NO: 1 comprises nt +101 to −200, +101 to −600, or +101 to −1000.

15-16. (canceled)

17. The plasmid according to claim 8, further comprising a nucleic acid sequence encoding a selection marker, a bacterial origin of replication, or a second nucleic acid sequence to direct integration of the exogenous DNA molecule into the genome of the type I alveolar epithelial cells.

18-19. (canceled)

20. The plasmid according to claim 17, wherein the second nucleic acid sequence to direct integration is a viral integration sequence.

21. An isolated host cell comprising the plasmid according to claim 9.

22. A composition comprising a pharmaceutically acceptable carrier and a plasmid according to claim 9.

23. The composition according to claim 22, wherein the carrier comprises an aqueous saline solution, a nanoparticle formulation, or a polymer.

24-25. (canceled)

26. The composition according to claim 23, wherein the polymer is a poly(ester amine) or a poly(amido amine).

27. A method of targeting an exogenous DNA into nuclei of type I alveolar epithelial cells, the method comprising:
providing a plasmid according to claim 9; and
introducing the plasmid into the cytoplasm of type I alveolar epithelial cells, wherein the nuclear targeting molecule targets the exogenous DNA into the nuclei of the type I alveolar epithelial cells.

28. The method according to claim 27, wherein said introducing is carried out by administering the plasmid into the lungs of a mammal in a manner effective to cause cells in the lungs to take up the plasmid.

29. The method according to claim 28, wherein the plasmid is simultaneously introduced into the cytoplasm of type II alveolar epithelial cells, but the nuclear targeting molecule does not target the exogenous DNA into the nuclei of the type II alveolar epithelial cells.

30. The method according to claim 27, wherein the plasmid is present in a composition further comprising a carrier.

31. The method according to claim 30, wherein the carrier comprises a nanoparticle formulation, an aqueous saline solution, or a polymer.

32-33. (canceled)

34. The method according to claim 31, wherein the polymer is a poly(ester amine) or a poly(amido amine).

35. The method according to claim 27, wherein said introducing further comprises exposing the lungs of the mammal to an electric field or ultrasound.

36. (canceled)


SEQUENCE LISTING (3 sequences)

SEQ ID NO: 1 - DNA, 1352 bp, Rattus norvegicus
gaattcggccttgcgcttcctaggtaagcgctctaccactgagctaaatccccagccccaacttattttttttttttacattgtgtgtgtgtgtgtgtctgtgtgtgtgtgtctgtgtgtgtctgtgtgtgtgtgtgtacatgtgtgagctgtctggtcgagagggcaacattcaggaatcaggtctctcatttcaccattgtgggtcagagcatagaactcaagcactaggcttgatgactaagccttctctgggagtggggaattggggggggggggaatcttcagatgcttttgtcgaaaggaatttatggcatctgggagtggagccgagaccacccccaaccctgctcttaagttccagcaggttagaagggaacacttgcttatacccactacctgagagcaaggaaggatttttttttttttttaaccaagagacggggcattaccagctaaaccctaggattaaaatagatgagctgaactcttagttccaggtgagtggctgctagtcaggagacacagctgctcccgtctccaggtgaagccacagtgtcccagtatcaggaggtctcatggtctctccgtttcaaatctgtgtaagtcgctagggccaagaactcttcaaggtcaaacaccccacccagacaagtacctcacaaagccaagtactgcactgggggccagctgtgggcaggacaggcacccacatccatgcatcacgtgcctggatgagggactcagttaattaagccgagccgcctcagactcacacaaacaacggctgttttccttaaaccatcagattcaatatttacaaaaaaattaaaaagccaatgcgtcgtcctaaaaccataaaaccagccgcacaggccactgtgtgagtctcccttctgcccagtaacaagaaaacctaagtcgtgtgccttgcccttcaaagcagaatagtaaagatagtagaaaaataaataaaaattgtaaaacagaactgtgtattgcatctgtcccccctaccccccttctcctgtagtggaacaaagtcaaacgctttttttttttttttttttttggcctgggctgggcccccagtggctgaggccctgtaactttaaacctggcctgaggtcatcttgtgatgctggccaaacagaattcgggggcggtcctagatctccgggaccacctgacgcccacccgcccccgcccccgtactggagacataaattgccgactgtgctgagaagttgccagttgccaagtttgctgtgcttgctccctccgctcgccttgaaagcccgggcactctctgtcgctgggactcttgctcaccgcttgccagcctcctcaga

SEQ ID NO: 2 - DNA, 2077 bp, Homo sapiens
atccactaactccttcatcctggcccctctccccaccccaggacccccagctgaatgcaattccttattacctcccactcctccatcctgatcaaaaggtcatgtgaccaaccttctagattctagacacatccgggggtgtgggatagaaggcaggtgtccttgaatcatgacctagcctcagcatccctctaaattagttgcatgtaacccaatctcccccacctaggcttcactgtgtttcaatctttttgaataaagcgggccatctttctttctgtgtgtgaccttggagttacacataagcagaatagatgtgcatctattctccatctaacaggctcaacagacaatgcactattcctcctgtcaacagacattattgacactagagtcataaaatgcatctccaagatggggaagtcagttcaagaaagagtatctgtatcacgagttttctctttacatttccaactccttggctctaggggtgttgccgctacccctcatcttctaggttcaggtgctcagctcctgtttgtagccccaggacaagacgttacctgggagatctttagaaatgcagaatctctccctatcccacaacagaattgcacctgcataaccagtgccccaggtagatctggctgagaagctatgatgaagggcaaggtttgccaaatgttcatggggacaagttctccagcgacactggtttaaaataggaattccgaaaaggtctgattaatgagtttggggtggaacccaggaaactgggcatttatttaaaacaatctctcctgtgattcttagaaagtaaatttataatggggaggggtcaaagataagcatctgaaaacaattttcacgcaatgtggagctttgagacagcgttagtcttcaggttcttttgttgaatggaatttgtggcatctgggagtggagcagagaccacttcccaaactcctgttcttatttttttaaattccagcaattcagaaggaattaaagtaaaagacgtaagttctctggttgctgatatctggtacctgagatcaaggcaagatcgcttaaaccagtaaagtgaggattatgggagcaaactcaggaataaagagggtgaaccaggcctggaaggggacacaggtttggcgaccagacgggtcaccgcttggcccggcctaatccaactgcgcacgtcggtgggccacagcatcgcagcgctggatccccgggtcggcagaggcggatggagttcagcctctttttcaaaactgccaaagccacccggtctgccaaactcccccaccagccagctctggggcagccccccggagcccgcccgctccggtccccgggacaagggcgcaattgaccagcgcgggccgcctctggcccacccagatgacggccaactttttttgtttttaagccatcaaattcaatatttacaggaaaaaccccatgcttggttctaaaactactggccactcaagaaacagtttacttctcacttctgcccataacaagaaaaacatagacttgtgccttactcttaaaaaaataataataataaatataggttttttaaaatttatatataatttttaaaaccccgtttgttgcatgtggtccctttccctcctgttgttgagcagaataaaagcccattttgccagggcccgcagtcctggcggcccccgcagctgcggccctgtaactttaaacctggcctgagatcatcgttttggcgccggccaaacagagcccgagggcgggcctagcgcgcccggacggagaccaccttgcggccgaccccgctcccccgcctcctcgggagagataaatgctgactccgctcggaaagttctcaactgcaaagtttgctgtccggctgcctagggtctgggaagctcgggcaccctccctctccggggctcctgctcccacccctccggcccccccaccgtcgcgctcctccaggctgggcctgtggccgcggtgctttttaattttcccccagctcagaatcttgctgctcggcccccaggagagcaacaactcaacgggaacgatg

SEQ ID NO: 3 - DNA, 1838 bp, Rhesus macaque
gtgtttcaatcttttaaataaagcaggccatctttctttctgagtgtgaccttggagttacacatacgcaaaatagatttgcatctattcttcatctaacaggctcaacagacaatgcacagttcctcctgtcaacagacattattgacactagagtcataaaatgcatctccaagatggggaagtcagttcaagaaagattatctgtatcactagttttctccttccatttccaaccccttggctctaggggtgttgcccccacccctcatcttcttggttcaggggctcagctcctgtttgtagcctcaggacaagatgttaccagggagatctttagaaatgcagaatctctcactaccccacaacggaattgcacctgcataaccaacgccccaggtagatccggctgagaagctatgatgaagggtaaggtttaccaaatgttcctggggacaagttctcctgcgacactggtttaaactaggaattcccaaaaggtctgattaatgagtttggagtggaatccaggaaactgggtatttaaaacaatctctcctgcgattcttagatagtaaatttataatggggaggagtcaaagataagcatctgaaaacaattttcacccagtgtggcgcttcgagacagcgttacgttcttttgttgaaaggaatttgtggcatctgggagtggagcagagaccacttcccaaactcctgttcttcttatttatttattttttttaattccaacaattcagaaggaattaaagtaaaagacgtaagttctccggttgctgatatctggtacctgagatcaaggcaagattgcttaaaccagtaaagtgaggattatgggggcaaactcaggaataaagagggtgagctcggcctggaaggggacacaggtttggcgaccagatgggtcaccaattggcccggcctaatccaactgcgcacgccggtgggccacagcatcgcagggctggatctccgggtcgggagaggcggatggaattcagcctctgtttcaaaactgccaaagccacccgctctgccaaactcccccaccagccagctctgggacagccctccggagcccacccgctcccgtccctgggacaagggcgcaattgaccagcgcgggcagcctccggcccacccagatgacggccaactttttttttttttaagccatcaaattcaatatttacaggcacaaccccatgcttggttctaaaactactggccactcaaggaacagtttacttctcacttctgcccataataagaaaaacagacttgtgccttgctcttaaaagaaaaaaaaaagaataataaatataggtttttaaaaatttatatataattttttaaaccccgtttgttgcatgtggtccctttccctcctgttgctgaacagaagaaaagcctattttgccagggcccacagtcctggcggcccccgcagctgcggccctgtaactttaaacctggcctgagatcatcgttttggcgccggccaaacagagcccgagggcgggcctagcgcgcccggacggagaccaccttgcggccgaccccgctcccccgcctcctcgggagagataaatgctgactccgctcggaaaagttctcaactgcaaagtttgctgtccggctgcctgaggtctgggaagctcgagcaccctcccgcgccggggctcctgcttccaccgctccggcccccaccgtcgcgctcctccaggctgggcctgtggccgcggcgctttttaatcttcccccagcgcagaaacttgctgctcggcccctaggagagcaactcaacgggaacgatg